(19) United States
(12) Patent Application Publication
(10) Pub. No.: US 2007/0112421 A1
(43) Pub. Date: May 17, 2007

(54) Medical device with a grooved surface

(76) Inventor: Barry O'Brien, Barna (IE)

Correspondence Address: JONES DAY, 222 EAST 41ST ST, NEW YORK, NY, 10017, US

(21) Appl. No.: 11/274,793

(22) Filed: Nov. 14, 2005

(51) Int. Cl. A61F 2/06

(52) U.S. Cl. 623/1.46

(57) ABSTRACT

This invention relates generally to medical devices, such as stents, for providing a medical treatment to an area of a patient, such as a body lumen. More particularly, the invention is directed to a stent comprising a grooved surface. The invention is also directed to a method for manufacturing a medical device comprising a grooved surface.

### FIELD OF THE INVENTION

This invention relates generally to medical devices, such as stents, for providing a medical treatment to an area of a patient, such as a body lumen. More particularly, the invention is directed to a stent comprising a grooved surface. The invention is also directed to a method for manufacturing a medical device comprising a grooved surface.

### BACKGROUND OF THE INVENTION

A variety of medical conditions have been treated by introducing an insertable medical device into a patient's body. Many of these devices having a coating, which may contain a therapeutic agent. For example, various types of medical devices are coated with a therapeutic agent, such a drug coated stent, have been proposed for treating an impaired body lumen. See, e.g., U.S. Pat. No. 6,099,562 to Ding et al. issued on Aug. 8, 2000. It is desirable that such devices, once implanted, are rapidly endothelialized to decrease the risks of thrombosis and restenosis that are associated with implanted devices in their pre-endothelialized state. In particular, it is a concern that in medical devices having a coating containing therapeutic agents, the therapeutic agents may inhibit endothelialization. Also, particularly in drug coated stents, it may be desirable to increase the surface area of a stent to increase rate at which the drug may be dispersed. Further it is a concern that current methods of achieving these ends may be uneconomical and may weaken the device structure.

It is therefore an objective of the present invention to allow for enhanced endothelialization of coated implanted medical devices while increasing surface area and maintaining strength and economical manufacture.

### SUMMARY OF THE INVENTION

A medical device is described comprising: a stent having at least a first strut, wherein the first strut comprises an outer surface configured to engage a body lumen when the stent is expanded, an inner surface opposite the outer surface, and at least one side surface extending between the outer surface and the inner surface; a coating disposed on a portion of at least one strut surface; and at least a first groove disposed in the coating and situated along the at least one strut surface.

The first groove may be situated on the at least one side surface. The first groove may extend between the outer surface and the inner surface. The stent may have a longitudinal axis, and wherein the first groove may be substantially perpendicular to the longitudinal axis of the stent. A plurality of grooves may be formed on the at least one side surface.

The coating may substantially conform to the at least one strut surface. The first groove may be formed on the inner surface of the first strut. The first groove may be formed on the outer surface of the first strut. The coating may have a substantially uniform thickness.

A second groove may also be formed along the at least one strut surface. The first groove and the second groove may have substantially different cross-sectional sizes. The first groove and the second groove may have substantially different cross-sectional shapes. The first groove and the second groove may have substantially similar cross-sectional sizes. The first groove and the second groove may have substantially similar cross-sectional shapes.

The first groove has a length, and wherein the cross-section of the first groove may vary along its length. The first groove may have a substantially triangular cross-section, a substantially rectangular cross-section, or a substantially U-shaped cross-section.

The coating may comprise a therapeutic agent. The coating may comprise a polymer.

A medical device is also described comprising: a stent having at least a first strut, wherein the first strut comprises an outer surface configured to engage a body lumen when the stent is expanded, an inner surface opposite the outer surface, and at least one side surface extending between the outer surface and the inner surface; a coating disposed on a portion of the at least one side surface, wherein the coating comprises a polymer and a therapeutic agent; and at least a first groove disposed in the coating and situated along the at least one side surface.

Another medical device is also described comprising: a stent having at least a first strut, wherein the first strut comprises an outer surface configured to engage a body lumen when the stent is expanded, an inner surface opposite the outer surface, and at least one side surface extending between the outer surface and the inner surface; wherein at least one strut surface comprises at least a first groove therein; and a coating disposed on a portion of the at least one strut surface in a manner that substantially preserves the groove.

Yet another medical device is described comprising: a stent having at least a first strut, wherein the first strut comprises an outer surface configured to engage a body lumen when the stent is expanded, an inner surface opposite the outer surface, and at least one side surface extending between the outer surface and the inner surface; wherein the at least one side surface comprises at least a first groove therein; and a coating disposed on a portion of the at least one side surface in a manner that substantially preserves the groove, wherein the coating comprises a polymer and a therapeutic agent.

A method for manufacturing a stent is described, comprising: (a) providing a stent comprising at least a first strut having a first surface; (b) coating at least a portion of the first surface with a coating; and (c) forming at least a first groove in the coating, wherein the first groove is disposed along the first surface.

The method may further comprise the step of coating at least a portion of a second surface of the first strut, and forming a second groove along the second surface. The first groove may be formed using physical abrasion, laser removal, and/or lithography. At least a portion of the coating may be selectively removed to form the first groove.

Another method for manufacturing a stent is described, comprising: (a) providing a stent comprising at least a first strut having a first surface; (b) selectively removing at least a portion of the first surface to form a first groove in the first surface; and (c) coating at least a portion of the first surface with a coating, wherein the first groove is substantially preserved after the coated has been applied. The coating may be substantially uniformly applied to the first surface.

Yet another method for manufacturing a stent is described, comprising: (a) providing a stent comprising at least a first strut having a first surface; (b) selectively depositing a coating on a first portion of the first surface and a second portion of the first surface, such that a groove is formed between the coating on the first portion and the coating on the second portion.

The selectively deposited coating on the first portion may have a first length, wherein the selectively deposited coating on the second portion may have a second length, wherein the groove may have a third length, and wherein the first and second lengths may be substantially greater than the third length. The first and second lengths may be substantially equal. At least one groove may be selectively deposited coating on the first portion.

### BRIEF DESCRIPTION OF THE DRAWINGS

Preferred features of the present invention are disclosed in the accompanying drawings, wherein similar reference characters denote similar elements throughout the several views, and wherein:

FIG. 1A is a top view of an exemplary stent with radially expandable cylindrical elements;

FIG. 1B is an oblique view of the stent of FIG. 1A in an unexpanded state;

FIG. 1C is an oblique view of an exemplary stent of FIG. 1B with radially expandable cylindrical elements connected by connecting elements in an expanded state;

FIG. 2A is a partial cross-sectional view of an exemplary strut of a stent deployed in a lumen;

FIG. 2B is a partial side view of a strut with side surface grooves deployed in a lumen;

FIGS. 3A-3D show an exemplary manufacturing process resulting in surface grooves;

FIGS. 4A-4C show another exemplary manufacturing process resulting in surface grooves;

FIGS. 5A-5D show yet another exemplary manufacturing process resulting in surface grooves;

FIG. 6 is a perspective cutaway view of an exemplary stent having grooves and deployed in a vessel;

FIGS. 7A-7F show exemplary struts having a variety of groove shapes;

FIGS. 8A-8D are perspective views of various groove arrangements on an exemplary strut;

FIGS. 9A-9H are cross-sectional views of various groove arrangements on an exemplary strut;

FIG. 10A is a top view, partially in section, of the stent in an unexpanded state within a body lumen, adjacent to a target tissue site;

FIG. 10B is a top view, partially in section, of the configuration of FIG. 10A, wherein the unexpanded stent is positioned at the target tissue site;

FIG. 10C is a top view, partially in section, of the configuration of FIG. 10B, wherein the stent is expanded and the struts are in contact with the target tissue site; and

FIG. 10D is a top view, partially in section, of the configuration of FIG. 10C, wherein the delivery catheter is withdrawn and the stent is fully expanded;

### DETAILED DESCRIPTION OF THE INVENTION

The invention described in detail herein generally relates to a medical device having at least one groove, and more particularly relates to a stent having a strut having at least one groove, though other medical devices are expressly contemplated, and will be appreciated by those skilled in the art. Suitable stents include ones that are used for cardiovascular and other medical applications. Other suitable stents include, for example, intravascular stents such as those described in U.S. Pat. No. 6,478,816 to Kveen et al, for “Stent”, issued on Nov. 12, 2002, incorporated herein by reference in its entirety. Suitable stents also include self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. Other suitable medical devices include heart valves and septal occluders.

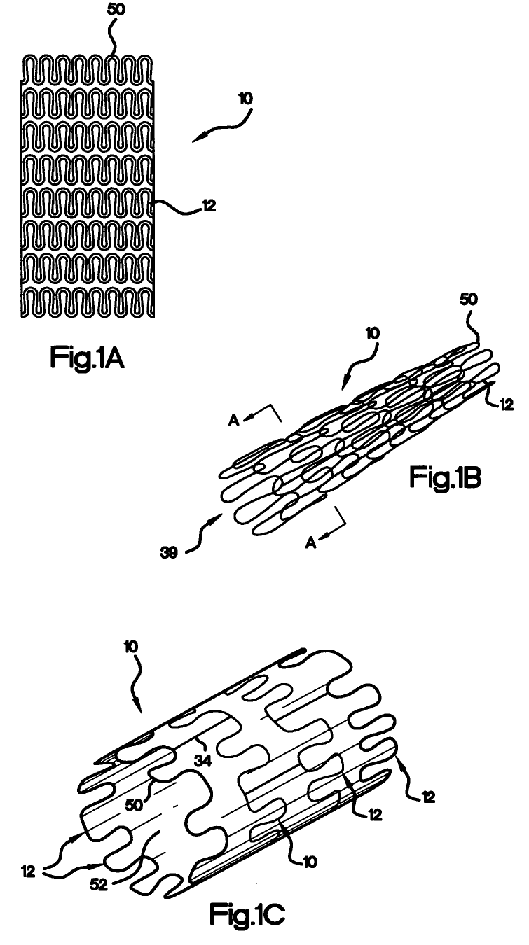

FIGS. 1A-1C show exemplary embodiments of a stent 10 that is suitable for use in the present invention. The stent 10 may have a flow path 39 therethrough. Stent 10 may also comprise a plurality of radially expandable cylindrical elements, and further may generally comprise struts 50 having a “peak” and “trough” configuration to form alternating loops. Adjacent radially expandable cylindrical elements 12 may be formed if at least two struts 50 are be connected to at least one connecting element 34.

Stents that are suitable for the present invention may be fabricated from metallic, ceramic, or polymeric materials, or a combination thereof. Metallic materials are more preferable. Suitable metallic materials include metals and alloys based on titanium (such as nitinol, nickel titanium alloys, thermo-memory alloy materials), stainless steel, tantalum, niobium, iridium, platinum, nickel-chrome, or certain cobalt alloys including cobalt-chromium-nickel alloys such as Elgiloy® and Phynox®. Metallic materials also include clad composite filaments, such as those disclosed in WO 94/16646.

Suitable ceramic materials include, but are not limited to, oxides, carbides, or nitrides of the transition elements such as titanium oxides, hafnium oxides, iridium oxides, chromium oxides, aluminum oxides, and zirconium oxides. Silicon based materials, such as silica and silicon nitride, may also be used.

The polymer(s) useful for forming the stent should be ones that are biocompatible and avoid irritation to body tissue. They can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for stents include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(γ-caprolactone), poly(γ-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of cells and molecules, such as proteins, nucleic acids, and the like.

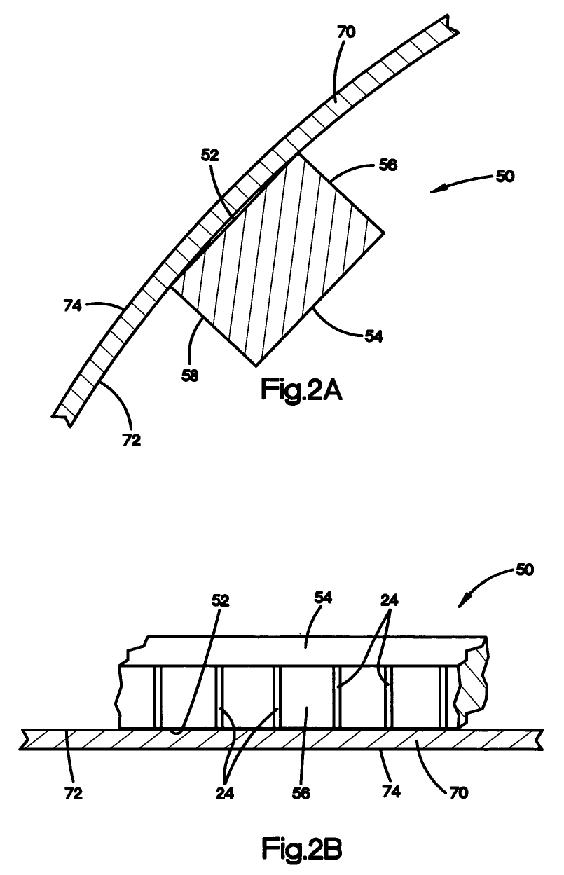

FIG. 2A shows an exemplary cross-sectional view of a stent strut 50 adjacent an inner surface 72 of a vessel 70. Strut 50 may have a plurality of surfaces, namely an outer surface 52, an inner surface 54, and side surfaces 56, 58. Typically, at least the outer surface 52 of a strut 50 contacts an inner surface 72 of a vessel 70 when a stent is expanded. Accordingly, inner surface 54 and side surfaces 56, 58 may not contact the vessel 70 upon initial expansion. Vessel 70 may also have an outer surface 74.

FIG. 2B shows a stent 10 having grooves 24 located on a first side surface 56 of strut 50 and running from the edge of the inner surface 54 of strut 50 to the outer surface 52 of strut 50. As discussed in more detail below, the grooves 24 may be of a variety of cross-sections, lengths, orientations, frequencies, and locations and may follow a variety of paths. Further, each individual groove 24 may vary in its cross-section or size along its path. Moreover, a single strut 50 may have a single groove 24, two grooves 24, or any suitable number of grooves 24. Similarly, a single stent 10 may comprise one or more struts 50 having grooves 24. Any or all of the outer surface 52, inner surface 54, and side surfaces 56, 58 may have one or more grooves 24. A more detailed discussion of grooves 24 appears infra.

Grooves 24 may be formed on a surface of a strut 50 in a variety of manners. In the following examples, side surface 56 is sometimes used as an exemplary surface for demonstrating how grooves 24 may be formed. However, it is expressly contemplated that any or all of the strut surfaces herein described and contemplated may have grooves 24 as a result of any or all of the processes described herein. Multiples processes may also be utilized on a single stent 10, or a single strut 50.

As used herein and throughout, the term “groove” is not limited to elongate, or channel-like features. Grooves 24 expressly include depressions, indentations, pores, holes, channels, impressions, imprints, prints, stamps, marks, and other features with a dimension have an elevation lower than that of the surrounding surface. Grooves 24 may be of a variety of shapes, including elongate, circular, square, rectangular, irregular, polygonal, triangular, pyramidal, cylindrical, spherical, bowl-like, channel-like, and other suitable shapes that will be appreciated by those of skill in the art. All embodiments of grooves 24 appearing in the figures herein are exemplary, as numerous variations are expressly contemplated.

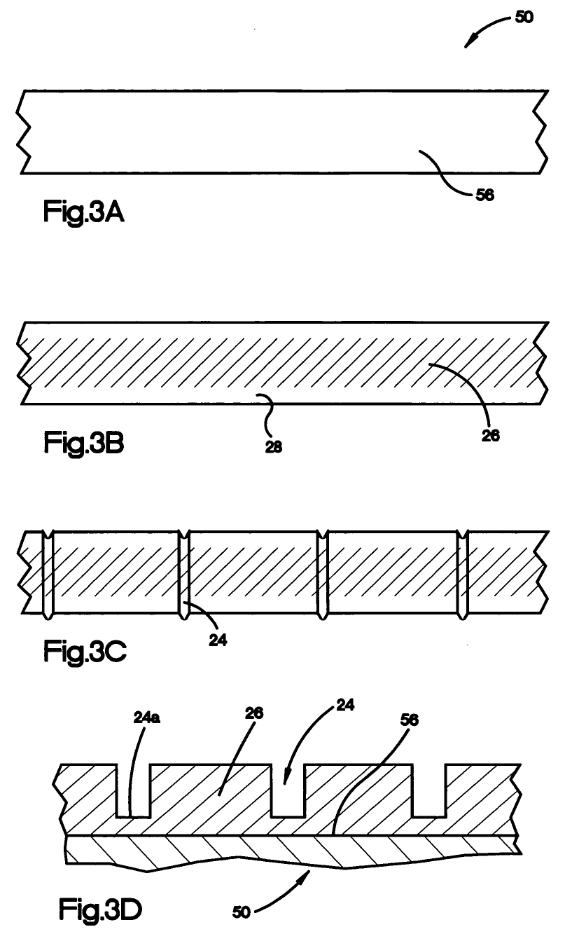

FIGS. 3A-3C show a view of the side surface 56 of strut 50 of an exemplary stent 10 in manufacture, wherein the strut 50 may go from an uncoated condition (FIG. 3A), to a coated condition (FIG. 3B), to a grooved condition (FIG. 3C).

FIG. 3B shows the strut 50 of FIG. 3A after a coating 26 has been applied. Coating 26 may contain a therapeutic agent and/or a polymeric material. A more detailed discussion of coatings 26 appears below. The coating 26 may have an outer surface 28 after it is applied to a strut 50. Moreover, coating 26 may be applied to strut 50 in a variety of thicknesses, layers, and/or patterns. For instance, coating 26 may be selectively applied to one or more of the inner 54, outer 52 or side surfaces 56, 58 of the strut 50. Coating 26 may be applied thicker on, or only to the grooves 24. Coating 26 may be applied thicker on and near the side surfaces 56, 58. Coating 26 may be applied in a single layer, or in multiple layers.

FIG. 3C shows the strut 50 of FIG. 3B in a grooved condition. Grooves 24 are formed in the coating 26. In one embodiment, the coating can be selectively removed to form the grooves 24 in the coating. Selective removal of the coating 26 may be undertaken by a number of techniques including fine mechanical or chemical abrading, chemical, laser or mechanical etching, or lithographic processes or any other processes known to one of skill in the art. In other embodiments, the grooves are formed by printing or forming impressions in the coating.

FIG. 3D shows an enlarged partial side view of the strut 50 of FIG. 3C. As is seen in this embodiment, grooves 24 are formed,in the coating 26, such that the base 24a of the groove is separate from the side surface 56 of the strut 50.

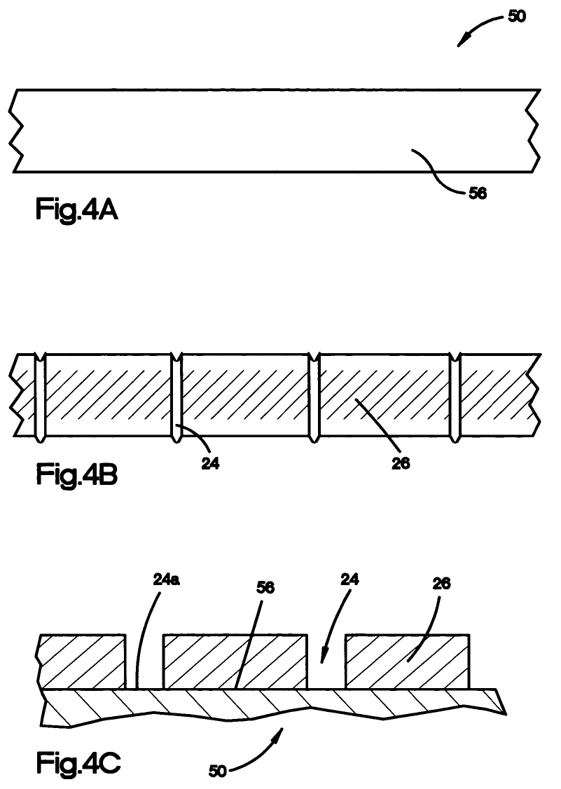

FIGS. 4A-4B show another embodiment of a mode of manufacture of a strut 50 of an exemplary stent 10 wherein the strut 50 may go from an uncoated condition (FIG. 3A), to selectively coated, grooved condition (FIG. 4B).

FIG. 4A shows a view of the side surface 56 of strut 50 of an exemplary stent 10. FIG. 4B shows the strut 50 of FIG. 4A after a coating 26 has been selectively applied to some portions of the strut. Grooves 24 may be created in the vacant space between two adjacent sections of coating. Selective coating may be achieved through such processes as printing, masking, and/or spraying. Coating 26 may contain a therapeutic agent and/or a polymeric material. A more detailed discussion of coating 26 embodiments appears below.

FIG. 4C shows an enlarged partial side view of the strut 50 of FIG. 4B. As seen in this embodiment, base 24a of grooves 24 is concurrent with side surface 56, and coating 26 does not extend completely along the shown portion.

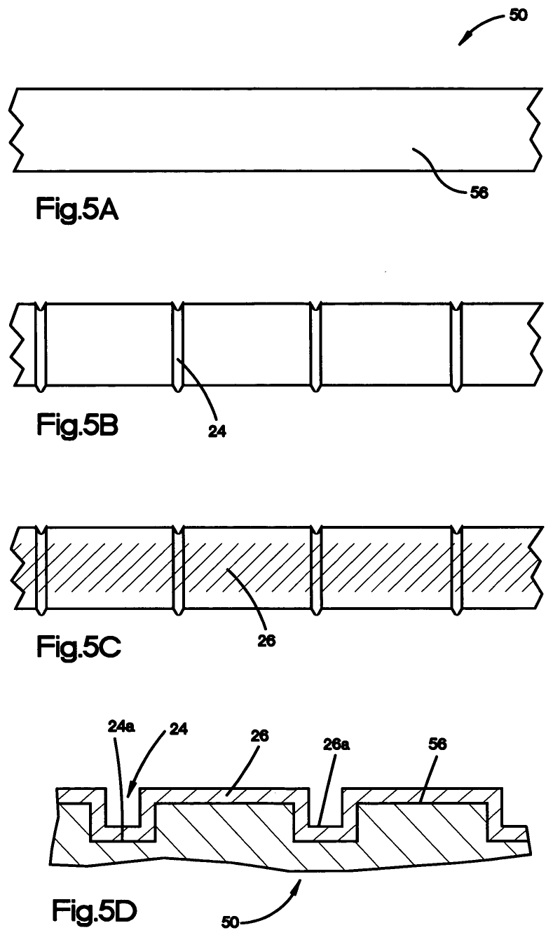

In yet another embodiment, FIGS. 5A-5C show a strut 50 of an exemplary stent 10 in manufacture, wherein the strut 50 may go from an uncoated, ungrooved condition (FIG. 5A), to an uncoated, grooved condition (FIG. 5B), to a coated, grooved condition (FIG. 5C).

FIG. 5A shows a view of a side surface 56 of strut 50 of an exemplary stent 10. FIG. 5B shows the strut 50 of FIG. 5A wherein grooves 26 running between inner surface 54 and outer surface 52 has been formed in the strut 50. These groove can be formed by selective removal of material from the strut. Removal may be undertaken by a number of techniques including fine mechanical abrading, chemical, laser or mechanical etching, printing or lithographic processes or any other tool or process known to one of skill in the art. Alternatively, grooves 24 may be created by selective deposition of material on sites adjacent to the groove 24 locations.

FIG. 5C shows the strut 50 of FIG. 5B after a coating 26, preferably a substantially uniform coating, has been applied to the groove 24 location and surrounding area thereby forming a stent 10 with a strut 50 with a coated surface and at least one groove 24. Coating 26 may generally conform to the shape of grooves 24, and may have a substantially constant thickness along the surface 56 of the stent 50. As discussed above and in greater detail below, coating 26 may contain a variety of substances and therapeutic agents and may be applied in a number of means and take a number of forms.

FIG. 5D shows an enlarged partial side view of the strut 50 of FIG. 5C. In this embodiment, the base 24a of the grooves 24 is separate and distinct from the surface 26a of the coating 26, but because the coating 26 generally conforms to the shape of the grooves 24, the groove 24 shape is preserved after the coating 26 has been applied.

Different coating methods may be used to vary the shape of the groove produced. For example, FIGS. 5A-5C show a substantially uniform coating that will produce grooves substantially similar to the underlying groove in the strut. However, one of skill in the art will recognize that varying the ways in which coating is applied, will vary the coating thickness and alter the shape of the groove produced.

The presence of grooves 24 in a strut 50 surface, particularly an inner surface 54 or side surface 56, 58, may be beneficial in encouraging endothelial cell migration from the abutting arterial wall to up and around the stent strut. This may be especially true with grooves 24 located on side surfaces 56, 58, as grooves 24 located on such surfaces 56, 58 may be, upon expansion of a stent 10 in a body lumen, situated adjacent to endothelial cells. This arrangement may result in an acceleration of endothelial cell migration as side surface 56, 58 grooves 24 may provide an easily accessible path for endothelial cells to “migrate up” the strut 50, and eventually encapsulate the stent 10 as a whole. Further presence of grooves 24 may also increase the surface area of the stent 10, and consequently may allow therapeutic agents in the stent coating 26 to be dispersed more rapidly into the blood stream. It should also be noted that certain placement of certain shapes, sizes, and patterns of grooves 24 directly in a strut 50 structure may undesirably weaken the stent 10 and decrease the fatigue performance of the stent 10. Locating the grooves in the stent 10 coating 26, rather than in the underlying strut 50, may prevent such a loss of strength and fatigue performance.

FIG. 6 shows a partial cutaway view of an exemplary stent 10 strut 50 with grooves 24 deployed in a vessel 70, and engaging the inner wall 72 of the vessel 70. As seen in FIG. 6, the inner wall 72 of the vessel may have an endothelial cell layer 76. As discussed herein in detail, grooves 24 positioned on the surface of a strut 50, preferably a side surface 56, 58, may promote the migration of endothelial cells into the grooves 24. Referring to FIG. 6, the movement of endothelial cells from the endothelial cell layer 76 to the grooves 24 may be demonstratively shown by the arrows extending from the endothelial cell layer 76 toward grooves 24.

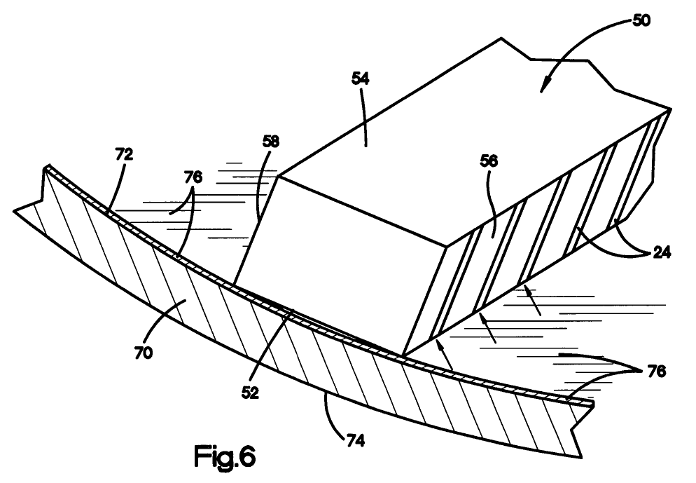

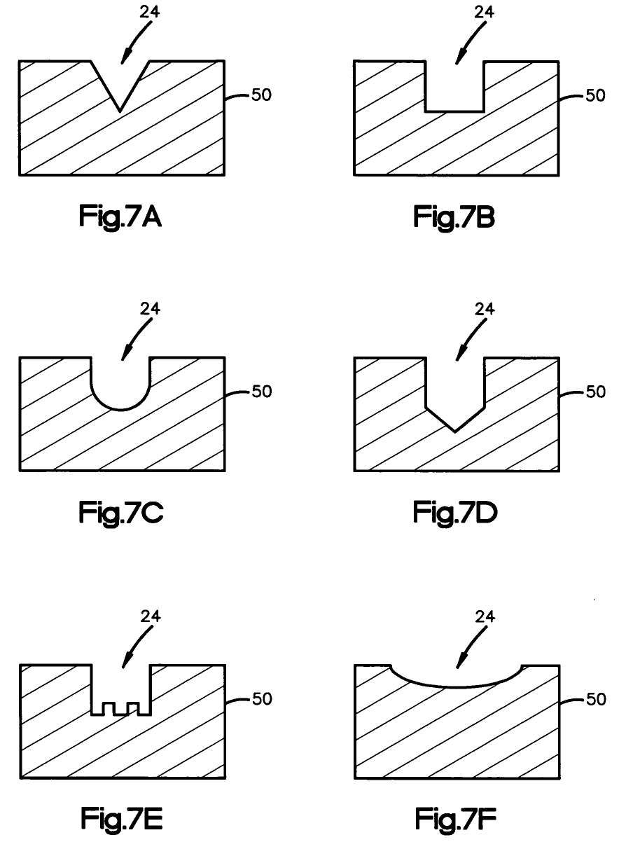

FIGS. 7A-7F show a variety of groove 24 cross-sections. Specifically, a groove 24 may have a triangular cross-section (FIG. 7A), a rectangular cross-section (FIG. 7B), a U-shaped cross-section (FIG. 7C), a polygonal-shaped cross-section (FIG. 7D), a micro-grooved cross-section (FIG. 7E), or a shallow bowl-shaped cross-section (FIG. 7F). These shapes are meant to be exemplary, for variations and combinations of these and other shapes are expressly contemplated and will be appreciated by those skilled in the art. A single strut 50 surface may have more than one groove 24 shape. A single groove 24 may have more than one cross-sectional shape and/or size. Such cross-sections may increase and/or decrease in cross-sectional area along a single groove 24. For example, a groove 24 with a semicircular cross-section may increase in cross-sectional area as it progresses from an outer surface 52 of a strut 50 to an inner surface 54 of a strut 50. As another example, a groove 24 may vary from a substantially semicircular cross-section from an outer surface 52 of a strut 50 to a substantially triangular cross-section at an inner surface 54 of a strut 50. Again, further variations and combinations are contemplated.

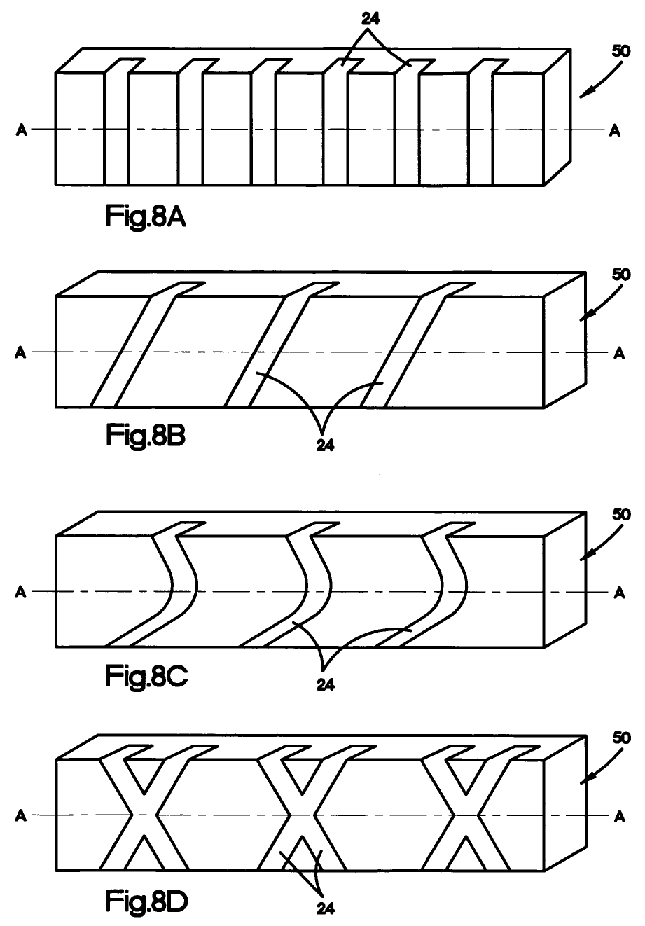

FIGS. 8A-8D show a variety of groove 24 patterns on a side surface 56 of a strut 50. Grooves 24 may take a variety of pathways to form a variety of patterns on the surface(s) of a strut 50. Grooves 24 may be substantially perpendicular to the longitudinal axis A-A of a strut 50 as shown in FIG. 8A. Grooves 24 may be angled relative to the longitudinal axis A-A of the stent as shown in FIG. 8B. Grooves 24 may also follow a substantially curved path along a surface of a strut 50 as shown in FIG. 8C. Combinations of different pathways may appear on a single strut. Grooves 24 may also intersect one another as shown in FIG. 8D.

Grooves 24 may traverse only a portion of the width of a strut 50 surface. Grooves 24 may have a variable depth. Part or all of the groove 24 may lie under the surface of the strut 10 or stent coating 26.

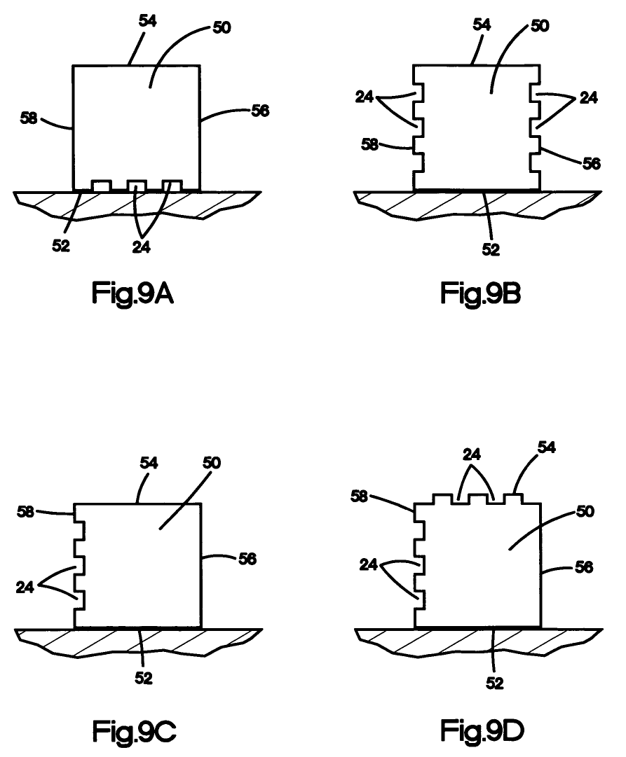

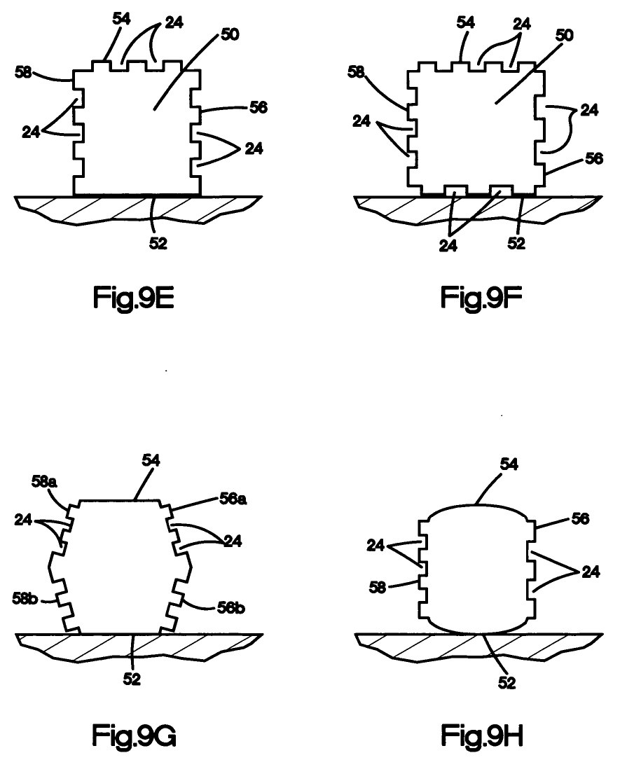

FIGS. 9A-9H show various arrangements and variations of a cross-sectional view of a strut 50 have at least one surface with grooves 24. For clarity, grooves 24 are shown in these illustrations as substantially parallel to the longitudinal axis of the strut 50. However, it is expressly contemplated that grooves 24 may be substantially perpendicular, angulated, or otherwise patterned in a variety of directions relative to the longitudinal axis of a strut 50.

As stated above, grooves 24 may be on one, some, or all of the surfaces 52, 54, 56, 58 of a strut 50. Specifically, grooves 24 may appear only on the outer surface 52 (FIG. 9A), on the side surfaces 54, 56 (FIG. 9B), on only one of the side surfaces 58 (FIG. 9C), on the inner surface 54 and one side surface 58 (FIG. 9D), on the inner surface 54, and both side surfaces 56, 58 (FIG. 9E), and on all surfaces 52, 54, 56, 58 of strut 50 (FIG. 9F).

A strut 50 may also have a variety of cross-sectional shapes, which may therefore provide further options for groove 24 placement and pattern. For example, as seen in FIG. 9G, strut 50 may have a substantially hexagon-shaped cross-section, which may have grooves 24 on side surfaces 56a, 56b, 58a, 58b. Strut 50 may also have a substantially oval cross-section, as seen in FIG. 9H, which may have grooves 24 on rounded side surfaces 56 and 58. These cross-sectional shapes are exemplary, as others are contemplated, including circular, rectangular, square, triangular, elliptical, polygonal, diamond-shaped, or any variation or combination of these and other shapes. Moreover, regardless of the cross-sectional shape of a strut 50, grooves 24 may or may not be placed on the variety of surfaces that may result from such a cross-sectional shape.

In a preferred embodiment, the width of the grooves may be from about 5 microns to about 100 microns and the depth of the grooves may be from about 2 to about 20 microns. More preferably, the width of the grooves may be from about 10 microns to about 50 microns, and the depth of the grooves may be from about 5 to about 10 microns.

In another embodiment, the grooves 24 may be seeded with endothelial cells, with the groove 24 profile acting to protect the seeded cells during deployment into the patient. Alternatively the grooves 24 could be used to contain some other biological, gene, or therapeutic agent.

In a further embodiment, grooves 24 may contain some agent with a therapeutic benefit. Placement of agents in the grooves 24 may protect the agents during manufacture, handling and once implanted in the patient. Further placement of agents in the grooves 24 may alter the rate and manner at which they are dispersed.

As discussed herein, grooves 24 may be formed by a variety of methods and materials. Grooves may be formed over the entire coating of the medical device or over only certain regions of the coating on the medical device. In one embodiment, multiple techniques may be used to imprint multiple grooves on a single device. In one embodiment of the present invention, grooves are formed on a coated medical device using a dimethylsiloxane (PDMS) mold with a pattern.

Grooves formed on the coating may be uniform or random. In one embodiment, grooves are uniformly formed on one section of the coating and randomly formed on another section of the coating. In another embodiment, the grooves are uniformly formed over the entire coating. In another embodiment, grooves are randomly imprinted on the coating.

The device may be formed with any shaped groove. The pattern may be smooth, without sharp edges or corners. The groove may be deep or shallow. In one embodiment, the grooves are orthogonal. A groove may be comprised of polygons such as circles, triangles, squares, shapes with regular or irregular sides and angles, or a combination thereof. In another embodiment, grooves comprise three-dimensional polygons.

The surface morphology of the device can be engineered to target a specific location of the body or in order to regulate the rate at which a biologically active material is released into the body. For example, in one embodiment, grooves are only formed to a first portion of the medical device. This type of groove formation may increase the surface area of the first portion. This embodiment may improve the localization of drug delivery by increasing the rate of drug release into the lumen while maintaining the same drug release rate in the blood.

Manipulating surface morphology may also allow for drug release rates on the ends of the medical device to be the same as the drug release rates in the middle of the medical device. In one embodiment, densely grooved portions may be formed in the middle of the device while more loosely grooved portions may be formed on the ends of the device. Since the edge of a device has more surface area over a given length than a face of the device, this groove formation may keep the drug release rate constant.

By forming a coating with different grooves, a very wide variety of surface areas can be achieved. The types of topology on a given region of the coating can be further varied by forming grooves an additional time.

As discussed herein, various techniques for forming grooves may be utilized. One type of exemplary technique involves removal of material from the coating or strut to form a groove. Examples of such techniques include without limitation fine mechanical or chemical abrading; chemical, laser or mechanical etching, printing, vapor deposition, or lithographic processes.

Suitable lithography techniques may include proximal probe lithography, scanning probe lithography or a combination thereof. In one embodiment of the invention, scanning probe lithography is used for forming grooves in the medical device coating with features smaller than 100 nm, 50 nm, 10 nm, 1 nm, or less. In one embodiment, scanning probe lithography is used to form grooves in the medical device coating with mechanical patterning such as scratching, nano-indentation, or local heating with a sharp tip. In another embodiment, grooves are formed in the coating using dip-pen nanolithography techniques.

Yet another process that may be used to form grooves are embossing techniques. Through recent advances in embossing, even nanoscale grooves can be formed through the embossing technique. In one embodiment of the invention, the embossing technique is used to form grooves in a coating that is too dried to be affected using either scanning probe lithography or printing techniques.

Also suitable for forming grooves are printing techniques. These printing techniques may include, but is not limited to, microcontact printing or inkjet printing, or a combination thereof. The microcontact printing method may use a polydimethylsiloxane (PDMS) or other elastomeric stamp to form the grooves. In one embodiment, the desired grooves can be formed on the stamp using conventional photolithography or another lithography technique. In another embodiment, microcontact printing is used to contemporaneously form grooves on every surface of the medical device that is in contact with the stamp at a given time.

Once the stamp is made, the grooves can be transferred to the coated medical device surface. By pressing the stamp into the coating before the coating is fully dry, the grooves on the stamp are formed in the coating. Preferably, the coating is not dried when the stamp is impressed into the coating. In one embodiment, the coating may be 70% to 100% dry when the stamp is imprinted onto the coating. In another embodiment, the coating may have rheological properties which enable the pattern to be retained on the coating while the coating is still malleable enough to be imprinted.

Another process that may be used to form grooves is a molding technique, which may include, but is not limited to, replica molding, microtransfer molding, micromolding capillaries, solvent-assisted micromolding, or a combination thereof.

The molding technique may use a polydimethylsiloxane (PDMS) or other elastomeric stamp to form grooves. In one embodiment, replica molding may be used to efficiently duplicate the information such as shape, morphology, and structure present on the surface of the coating. In another embodiment, replica molding may be used for duplicating two or three dimensional topologies on the coating of a medical device in a single step. Preferably, replica molding may enable the duplication of complex structures in the stamp in multiple copies of the coating with nanoscale resolution in a simple, reliable and inexpensive way. A single implementation of replica molding may be used multiple times on a single medical device, for a single time on the coatings of multiple medical devices, or for a combination thereof.

The size and shape of the stamp may be manipulated by controlled deformation of the stamp used to mold the pattern. By mechanically stretching, bending, compressing or a combination thereof, the surface of the stamp and thereby the pattern on the coating, can be inexpensively and reliably altered.

Microtransfer molding may be used to form grooves in a large surface of the medical device coating over a short period of time. In one embodiment, the coating of a medical device is molded with interconnected and isolated microstructures using microtransfer molding. In another embodiment, microtransfer molding is used in forming grooves where the coating of a medical device is nonplanar.

In one embodiment of the invention, microtransfer molding is used to form grooves in the form of arrays of parallel lines on the coating of a medical device. In another embodiment of the invention, geometric grooves that enhance endothelialization are formed on the device through microtransfer molding.

Micromolding in capillaries may also be used to form grooves on a medical device coating. In one embodiment of the invention, micromolding in capillaries is used to form nanoscale patterns on a medical device coating in a single step. In another embodiment of the invention micromolding in capillaries is used to create a freestanding microstructure out of the medical device coating, comprised of two interconnected layers, with an independent relief structure in each.

Solvent-assisted micromolding may be used to pattern the coating on a medical device in a single step. An elastomeric stamp, such as one fabricated with PDMS may be used. In one embodiment of the invention, solvent-assisted micromolding is used to create quasi-three-dimensional structures that are well defined and clearly resolved.

As discussed in detail above, it may be beneficial to apply a coating 26 to a stent 10 having struts 50. A coating composition may be prepared, for example, by applying a mixture of a therapeutic agent, solvent and/or a polymeric material on a surface to form a coating. If such a composition is used which includes a polymeric material, the polymeric material generally incorporates the therapeutic agent. Alternatively, the coating composition may not include a polymeric material. The following is a description of suitable materials and methods useful in producing a coating on the surface of stent struts of the invention.

Polymeric materials useful for forming the coating should be ones that are biocompatible, particularly during insertion or implantation of the device into the body and avoids irritation to body tissue. Examples of such polymers include, but not limited to, polyurethanes, polyisobutylene and its copolymers, silicones, and polyesters. Other suitable polymers include polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate; copolymers of vinyl monomers, copolymers of vinyl monomers and olefins such as ethylene-methyl methacrylate copolymers, acrylonitrile-styrene copolymers, ABS resins, ethylene-vinyl acetate copolymers, polyamides such as Nylon 66 and polycaprolactone, alkyd resins, polycarbonates, polyoxyethylenes, polyimides, polyethers, epoxy resins, polyurethanes, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, and polylactic acid-polyethylene oxide copolymers. Since the polymer is being applied to a part of the medical device which undergoes mechanical challenges, e.g. expansion and contraction, the polymers are preferably selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. The polymer is selected to allow the coating to better adhere to the surface of the strut when the stent is subjected to forces or stress. Furthermore, although the coating can be formed by using a single type of polymer, various combinations of polymers can be employed.

Generally, when a biologically active material used is a hydrophilic, e.g., heparin, then a matrix material comprising a more hydrophilic material has a greater affinity for the biologically active material than another matrix material that is less hydrophilic. When a biologically active material used is a hydrophobic, e.g., paclitaxel, actinomycin, sirolimus (RAPAMYCIN), tacrolimus, everolimus, and dexamethasone, then a matrix material that is more hydrophobic has a greater affinity for the biologically active material than another matrix material that is less hydrophobic.

Examples of suitable hydrophobic polymers include, but not limited to, polyolefins, such as polyethylene, polypropylene, poly(1-butene), poly(2-butene), poly(1-pentene), poly(2-pentene), poly(3-methyl-1-pentene), poly(4-methyl-1-pentene), poly(isoprene), poly(4-methyl-1-pentene), ethylene-propylene copolymers, ethylene-propylene-hexadiene copolymers, ethylene-vinyl acetate copolymers, blends of two or more polyolefins and random and block copolymers prepared from two or more different unsaturated monomers; styrene polymers, such as poly(styrene), poly(2-methylstyrene), styrene-acrylonitrile copolymers having less than about 20 mole-percent acrylonitrile, and styrene-2,2,3,3,-tetrafluoropropyl methacrylate copolymers; halogenated hydrocarbon polymers, such as poly(chlorotrifluoroethylene), chlorotrifluoroethylene-tetrafluoroethylene copolymers, poly(hexafluoropropylene), poly(tetrafluoroethylene), tetrafluoroethylene, tetrafluoroethylene-ethylene copolymers, poly(trifluoroethylene), poly(vinyl fluoride), and poly(vinylidene fluoride); vinyl polymers, such as poly(vinyl butyrate), poly(vinyl decanoate), poly(vinyl dodecanoate), poly(vinyl hexadecanoate), poly(vinyl hexanoate), poly(vinyl propionate), poly(vinyl octanoate), poly(heptafluoroisopropoxyethylene), poly(heptafluoroisopropoxypropylene), and poly(methacrylonitrile); acrylic polymers, such as poly(n-butyl acetate), poly(ethyl acrylate), poly(1-chlorodifluoromethyl)tetrafluoroethyl acrylate, poly di(chlorofluoromethyl)fluoromethyl acrylate, poly(1,1-dihydroheptafluorobutyl acrylate), poly(1,1-dihydropentafluoroisopropyl acrylate), poly(1,1-dihydropentadecafluorooctyl acrylate), poly(heptafluoroisopropyl acrylate), poly 5-(heptafluoroisopropoxy)pentyl acrylate, poly 11-(heptafluoroisopropoxy)undecyl acrylate, poly 2-(heptafluoropropoxy)ethyl acrylate, and poly(nonafluoroisobutyl acrylate); methacrylic polymers, such as poly(benzyl methacrylate), poly(n-butyl methacrylate), poly(isobutyl methacrylate), poly(t-butyl methacrylate), poly(t-butylaminoethyl methacrylate), poly(dodecyl methacrylate), poly(ethyl methacrylate), poly(2-ethylhexyl methacrylate), poly(n-hexyl methacrylate), poly(phenyl methacrylate), poly(n-propyl methacrylate), poly(octadecyl methacrylate), poly(1,1-dihydropentadecafluorooctyl methacrylate), poly(heptafluoroisopropyl methacrylate), poly(heptadecafluorooctyl methacrylate), poly(1-hydrotetrafluoroethyl methacrylate), poly(1,1-dihydrotetrafluoropropyl methacrylate), poly(1-hydrohexafluoroisopropyl methacrylate), and poly(t-nonafluorobutyl methacrylate); polyesters, such a poly(ethylene terephthalate) and poly(butylene terephthalate); condensation type polymers such as and polyurethanes and siloxane-urethane copolymers; polyorganosiloxanes, i.e., polymeric materials characterized by repeating siloxane groups, represented by Rₐ SiO_{4-a/2}, where R is a monovalent substituted or unsubstituted hydrocarbon radical and the value of a is 1 or 2; and naturally occurring hydrophobic polymers such as rubber.

Examples of suitable hydrophilic monomer include, but not limited to; (meth)acrylic acid, or alkaline metal or ammonium salts thereof; (meth)acrylamide; (meth)acrylonitrile; those polymers to which unsaturated dibasic, such as maleic acid and fumaric acid or half esters of these unsaturated dibasic acids, or alkaline metal or ammonium salts of these dibasic adds or half esters, is added; those polymers to which unsaturated sulfonic, such as 2-acrylamido-2-methylpropanesulfonic, 2-(meth)acryloylethanesulfonic acid, or alkaline metal or ammonium salts thereof, is added; and 2-hydroxyethyl (meth)acrylate and 2-hydroxypropyl (meth)acrylate.

Polyvinyl alcohol is also an example of hydrophilic polymer. Polyvinyl alcohol may contain a plurality of hydrophilic groups such as hydroxyl, amido, carboxyl, amino, ammonium or sulfonyl (—SO₃). Hydrophilic polymers also include, but are not limited to, starch, polysaccharides and related cellulosic polymers; polyalkylene glycols and oxides such as the polyethylene oxides; polymerized ethylenically unsaturated carboxylic acids such as acrylic, mathacrylic and maleic acids and partial esters derived from these acids and polyhydric alcohols such as the alkylene glycols; homopolymers and copolymers derived from acrylamide; and homopolymers and copolymers of vinylpyrrolidone.

The term “therapeutic agent” as used in the present invention encompasses drugs, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), pimecrolimus, amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other suitable therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

The solvent that is used to form the coating composition include ones which can dissolve the polymer into solution and do not alter or adversely impact the therapeutic properties of the therapeutic agent employed. Examples of useful solvents include tetrahydrofuran (THF), methyl ethyl ketone chloroform, toluene, acetone, issoctane, 1,1,1-trichloroethane, isoppropanol, IPA and dichloromethane or mixtures thereof.

Suitable stents may also be coated or made with non-polymeric materials. Examples of useful non-polymeric materials include sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; sphingomyelins such as stearyl, palmitoyl, and tricosanyl sphingomyelins; ceramides such as stearyl and palmitoyl ceramides; glycosphingolipids; lanolin and lanolin alcohols; and combinations and mixtures thereof. Preferred non-polymeric materials include cholesterol, glyceryl monostearate, glycerol tristearate, stearic acid, stearic anhydride, glyceryl monooleate, glyceryl monolinoleate, and acetylated monoglycerides.

In one method of forming the aforementioned coatings, a coating material composition is applied to the surface. Coating compositions can be applied by any method to a surface of a medical device to form a coating layer. Examples of suitable methods include, but are not limited to, spraying such as by conventional nozzle or ultrasonic nozzle, dipping, rolling, electrostatic deposition, and a batch process such as air suspension, pan coating or ultrasonic mist spraying. Also, more than one coating method can be used to make a medical device. Coating compositions suitable for applying a coating to the devices of the present invention can include a polymeric material dispersed or dissolved in a solvent suitable for the medical device, wherein upon applying the coating composition to the medical device, the solvent is removed. Such systems are commonly known to the skilled artisan.

A coating of a medical device of the present invention may include multiple coating layers. For example, the first layer and the second layer may contain different biologically active materials. Alternatively, the first layer and the second layer may contain an identical biologically active material having different concentrations. In one embodiment, either of the first layer or the second layer may be free of biologically active material. For example, when the biologically active solution is applied onto a surface and dried (the first layer), a coating composition free of a biologically active material (the second layer) can be applied over the dried biologically active material.

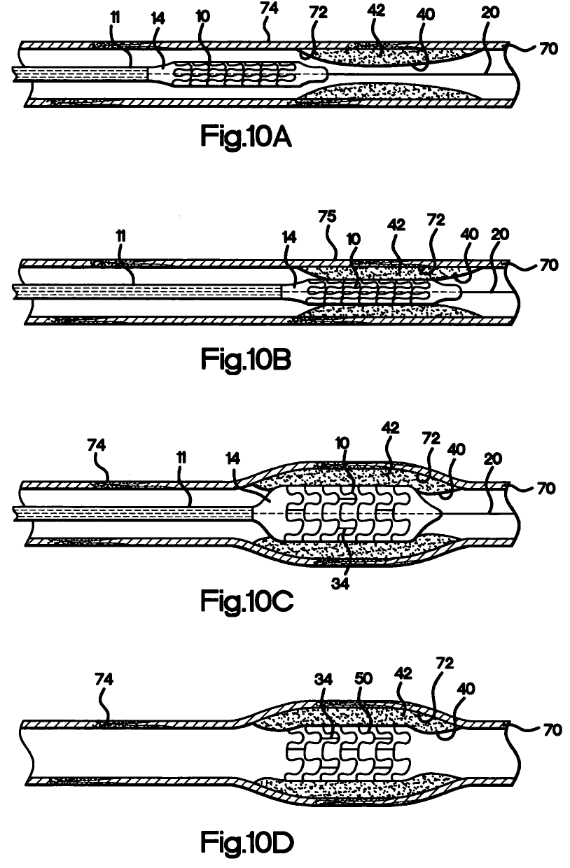

FIGS. 10A-10D show an exemplary delivery of a stent 10 into a body lumen. Stent 10 may first be mounted onto an inflatable balloon 14, or other mechanical delivery system, on the distal end of a delivery catheter 11. Stent 10 may be crimped or collapsed in substantially congruent dimensions to balloon 14. Guidewire 20 may be coaxially disposed in the body lumen prior to the introduction of the stent 10. Stent 10 and catheter 11 may then be introduced into a patient's body by methods such as the Seldinger technique, or other useful methods. Stent 10 and catheter 11 may be advanced over guidewire 20, at least to the area of obstruction 42. It may be preferable to advance the stent 10 until it is substantially centered in the area of obstruction 42.

When stent 10 is inserted into a desired location within a patient, balloon 14 may be inflated, which may thereby expand stent 10. At least one strut element 50 of stent 10 may thereby be brought into contact with at least a portion of the surface 40 of the obstruction 42 and/or the inner wall 72 of a vessel 70. Vessel 70 may be expanded slightly by the expansion of stent 10 to provide volume for the expanded lumen. As a result, interference of blood flow by stent 10 may be minimized, in addition to preventing unwarranted movement of stent 10 once the expansion is complete.

As stated above, stent 10 may be used coated or otherwise applied with a biologically active material. In certain embodiments, the biologically active material may be used to inhibit the proliferation, contraction, migration and/or hyperactivity of cells of the brain, neck, eye, mouth, throat, esophagus, chest, bone, ligament, cartilage, tendons, lung, colon, rectum, stomach, prostate, breast, ovaries, fallopian tubes, uterus, cervix, testicles or other reproductive organs, hair follicles, skin, diaphragm, thyroid, blood, muscles, bone, bone marrow, heart, lymph nodes, blood vessels, arteries, capillaries, large intestine, small intestine, kidney, liver, pancreas, brain, spinal cord, and the central nervous system. In a preferred embodiment, the biologically active material is useful for inhibiting the proliferation, contraction, migration and/or hyperactivity of muscle cells, e.g., smooth muscle cells.

In certain other embodiments, the biologically active material may be used to inhibit the proliferation, contraction, migration and/or hyperactivity of cells in body tissues, e.g., epithelial tissue, connective tissue, muscle tissue, and nerve tissue. Epithelial tissue covers or lines all body surfaces inside or outside the body. Examples of epithelial tissue include, but are not limited to, the skin, epithelium, dermis, and the mucosa and serosa that line the body cavity and internal organs, such as the heart, lung, liver, kidney, intestines, bladder, uterine, etc. Connective tissue is the most abundant and widely distributed of all tissues. Examples of connective tissue include, but are not limited to, vascular tissue (e.g., arteries, veins, capillaries), blood (e.g., red blood cells, platelets, white blood cells), lymph, fat, fibers, cartilage, ligaments, tendon, bone, teeth, omentum, peritoneum, mesentery, meniscus, conjunctiva, dura mater, umbilical cord, etc. Muscle tissue accounts for nearly one-third of the total body weight and consists of three distinct subtypes: striated (skeletal) muscle, smooth (visceral) muscle, and cardiac muscle. Examples of muscle tissue include, but are not limited to, myocardium (heart muscle), skeletal, intestinal wall, etc. The fourth primary type of tissue is nerve tissue. Nerve tissue is found in the brain, spinal cord, and accompanying nerve. Nerve tissue is composed of specialized cells called neurons (nerve cells) and neuroglial or glial cells.

The biologically active material, drug-eluting coatings, and coated medical devices of the present invention may also be used to treat diseases that may benefit from decreased cell proliferation, contraction, migration and/or hyperactivity, including, but not limited to stenosis and restenosis.

In particular, the biologically active material, such as paclitaxel, may be used to treat or prevent diseases or conditions that may benefit from decreased or slowed cell proliferation, contraction, migration or hyperactivity. In specific embodiments, the present invention inhibits or reduces at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, at least 10%, at least 5%, or at least 1% of cell proliferation, contraction, migration and/or hyperactivity.

The present invention further provides methods for treating or preventing stenosis or restenosis. In particular, the invention relates to methods for treating or preventing stenosis or restenosis by inserting or implanting a coated medical device of the invention into a subject.

As used herein, the terms “subject” and “patient” are used interchangeably. The subject can be an animal, preferably a mammal including a non-primate (e.g., a cow, pig, horse, cat, dog, rat, and mouse) and a primate (e.g., a monkey, such as a cynomolgous monkey, chimpanzee, and a human), and most preferably a human.

In one embodiment, the subject can be a subject who had undergone a regimen of treatment (e.g., percutaneous transluminal coronary angioplasty (PTCA), also known as balloon angioplasty, and coronary artery bypass graft (CABG) operation).

The therapeutically effective amount of a biologically active material for the subject will vary with the subject treated and the biologically active material itself. The therapeutically effective amount will also vary with the condition to be treated and the severity of the condition to be treated. The dose, and perhaps the dose frequency, can also vary according to the age, gender, body weight, and response of the individual subject. As used herein, the term “therapeutically effective amount” refers to that amount of the biologically active material sufficient to inhibit cell proliferation, contraction, migration, hyperactivity, or address other conditions (e.g., cancer). A therapeutically effective amount may refer to the amount of biologically active material sufficient to delay or minimize the onset of symptoms associated with cell proliferation, contraction, migration, hyperactivity, or address other conditions. A therapeutically effective amount may also refer to the amount of the biologically active material that provides a therapeutic benefit in the treatment or management of certain conditions such as stenosis or restenosis and/or the symptoms associated with stenosis or restenosis.

The present invention is useful alone or in combination with other treatment modalities. In certain embodiments, the subject can be receiving concurrently other therapies to treat or prevent stenosis or restenosis. In certain embodiments, the treatment of the present invention further includes the administration of one or more immunotherapeutic agents, such as antibodies and immunomodulators, which include, but are not limited to, HERCEPTIN®, RITUXAN®, OVAREX™, PANOREX®, BEC2, IMC-C225, VITAXIN™, CAMPATH® I/H, Smart MI95, LYMPHOCIDE™, Smart I D10, ONCOLYM™, rituximab, gemtuzumab, or trastuzumab. In certain other embodiments, the treatment method further comprises hormonal treatment. Hormonal therapeutic treatments comprise hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein by reference, in their entirety, for all purposes related to this disclosure.

While the invention has been shown and described herein with reference to particular embodiments, it is to be understood that the various additions, substitutions, or modifications of form, structure, arrangement, proportions, materials, and components and otherwise, used in the practice and which are particularly adapted to specific environments and operative requirements, may be made to the described embodiments without departing from the spirit and scope of the present invention. Accordingly, it should be understood that the embodiments disclosed herein are merely illustrative of the principles of the invention. Various other modifications may be made by those skilled in the art which will embody the principles of the invention and fall within the spirit and the scope thereof.

What is claimed is:

1. A medical device comprising:
a stent having at least a first strut, wherein the first strut comprises an outer surface configured to engage a body lumen when the stent is expanded, an inner surface opposite the outer surface, and at least one side surface extending between the outer surface and the inner surface;
a coating disposed on a portion of at least one strut surface; and
at least a first groove disposed in the coating.

2. The device of claim 1, wherein the first groove is situated on the at least one side surface.

3. The device of claim 2, wherein the first groove extends between the outer surface and the inner surface.

4. The device of claim 1, wherein the stent has a longitudinal axis, and wherein the first groove is substantially perpendicular to the longitudinal axis of the stent.

5. The device of claim 1, wherein a plurality of grooves are formed on the at least one side surface.

6. The device of claim 1, wherein the coating substantially conforms to the at least one strut surface.

7. The device of claim 1, wherein the first groove is formed on the inner surface of the first strut.

8. The device of claim 1, wherein the first groove is formed on the outer surface of the first strut.

9. The device of claim 1, wherein the coating has a substantially uniform thickness.

10. The device of claim 1, wherein a second groove is also formed along the at least one strut surface.

11. The device of claim 10, wherein the first groove and the second groove have substantially different cross-sectional sizes.

12. The device of claim 10, wherein the first groove and the second groove have substantially different cross-sectional shapes.

13. The device of claim 10, wherein the first groove and the second groove have substantially similar cross-sectional sizes.

14. The device of claim 10, wherein the first groove and the second groove have substantially similar cross-sectional shapes.

15. The device of claim 1, wherein the first groove has a length, and wherein the cross-section of the first groove varies along its length.

16. The device of claim 1, wherein the first groove has a substantially triangular cross-section.

17. The device of claim 1, wherein the first groove has a substantially rectangular cross-section.

18. The device of claim 1, wherein the first groove has a substantially U-shaped cross-section.

19. The device of claim 1, wherein the coating comprises a therapeutic agent

20. The device of claim 1, wherein the coating comprises a polymer.

21. The device of claim 1, wherein the first groove is substantially elongate.

22. A medical device comprising:
a stent having at least a first strut, wherein the first strut comprises an outer surface configured to engage a body lumen when the stent is expanded, an inner surface opposite the outer surface, and at least one side surface extending between the outer surface and the inner surface;
a coating disposed on a portion of the at least one side surface, wherein the coating comprises a polymer and a therapeutic agent; and
at least a first groove disposed in the coating.

23. A medical device comprising:
a stent having at least a first strut, wherein the first strut comprises an outer surface configured to engage a body lumen when the stent is expanded, an inner surface opposite the outer surface, and at least one side surface extending between the outer surface and the inner surface;
wherein at least one strut surface comprises at least a first groove therein; and
a coating disposed on a portion of the at least one strut surface in a manner that substantially preserves the groove.

24. The device of claim 23, wherein the first groove is situated on the at least one side surface.

25. The device of claim 24, wherein the first groove extends between the outer surface and the inner surface.

26. The device of claim 23, wherein the stent has a longitudinal axis, and wherein the first groove is substantially perpendicular to the longitudinal axis of the stent.

27. The device of claim 23, wherein a plurality of grooves are formed on the at least one side surface.

28. The device of claim 23, wherein the coating comprises a therapeutic agent

29. The device of claim 23, wherein the coating comprises a polymer.

30. A medical device comprising:
a stent having at least a first strut, wherein the first strut comprises an outer surface configured to engage a body lumen when the stent is expanded, an inner surface opposite the outer surface, and at least one side surface extending between the outer surface and the inner surface;
wherein the at least one side surface comprises at least a first groove therein; and
a coating disposed on a portion of the at least one side surface in a manner that substantially preserves the groove, wherein the coating comprises a polymer and a therapeutic agent.
